(19) United States
(12) Patent Application Publication
(10) Pub. No.: US 2009/0157112 A1
(43) Pub. Date: Jun. 18, 2009

(54) Magnetized acupuncture needle assembly having a magnetized acupuncture needle and method

(76) Inventor: Frank He, Sunnyvale, CA (US)

Correspondence Address: James Cal, Esq.;(Schein & Cai LLP), SUITE 1250, 111 W. St. John Street, SAN JOSE, CA, 95113, US

(21) Appl. No.: 11/543,751

(22) Filed: Oct. 6, 2006

(51) Int. Cl. A61B 17/34

(52) U.S. Cl. 606/189

(57) ABSTRACT

An acupuncture needle assembly with an acupuncture needle and needle guide tube which are constructed and arranged to adhere magnetically together. The magnetic attraction allows the acupuncture needle to be held in the needle guide tube automatically. The needle guide tube may include magnetic or ferrous particles or material in its body structure. Preferably, the acupuncture needle includes magnetic or ferrous particles or material in its body structure. The needle handle may also glow in the dark.

### BACKGROUND OF THE INVENTION

1. Field of the Invention

The present invention relates to acupuncture medical treatments and, more particularly, to a magnetized acupuncture needle assembly employing magnetic forces.

2. General Background

While acupuncture is a well accepted ancient eastern medical treatment, only recently has acupuncture been actively used alone as a healing modality or to supplement western medicine. During an acupuncture treatment, the professional inserts into the skin (body tissue) of a patient an acupuncture needle (a very thin cylindrical metal body with a sharp point on one end) at designated points of the anatomy. Thus, during the acupuncture treatment, the skin is pierced. Hence, good hygiene of the professional and the sterile needles are necessary to minimize, if not prevent, communication of bacteria, germs, or diseases to the patient.

As can be readily appreciated, minimizing direct contact with the sterile acupuncture needle and its sharp point would minimize communication of bacteria, germs and diseases. Various modes of inserting the acupuncture needle use a needle guide tube. One example of a guide tube 10A, is shown in FIGS. 1A and 1B. In this example, an acupuncture needle 12A is housed and secured in the needle guide tube 10A until used, as best seen in FIGS. 1A and 1B. However, to keep the acupuncture needle 12A from falling through the needle guide tube 10A, the acupuncture needle 12A was secured in the tube 10A by wedging the acupuncture needle 12A with a wedge member 14A, as best seen in FIG. 1B. The wedge member 14A forces the acupuncture needle 12A to one side of the tube 10A causing bends or other deformation in the acupuncture needle 12A, as it is compressed between the wedge member 14A and the interior surface of the tube 10A.

Bends and deformations in the acupuncture needle 12A can compromise the acupuncturist's ability to pierce the exact location intended to be pierced. Bends and deformations would skew any forces (such as the result of tapping) traveling along the length of the needle for piercing the body tissue.

Referring now to FIGS. 2A and 2B, alternately, the acupuncture needle 12B may include a handle 14B which can be wedged or squeezed into the needle guide tube 10B. The handle 14B of the acupuncture needle 12B may be secured inside the needle guide tube 10B. In this example, a portion 15 of the handle 14B is fatter or larger than the rest of the handle structure. Furthermore, the needle guide tube 10B has a rim or projection 11 in the interior thereof. Thus, when the acupuncture needle 12B is threaded into the needle guide tube 10B, the acupuncture needle 12B is held in the guide tube by the rim 11 because the portion 15 is larger than the opening through the rim 11. Thus, the rim 11 forms an obstruction, as best seen in FIG. 2A, for holding the acupuncture needle 12B and preventing it from falling through the needle guide tube 10B.

As can be appreciated, in this example, the use of the needle guide tube for inserting the needle to pierce the skin is cumbersome and achieving the proper penetration is often compromised.

Thus, there is a continuing need for a magnetized acupuncture needle assembly which is constructed and arranged to magnetically attract the acupuncture needle to the needle guide tube to automatically hold the acupuncture needle within the needle guide tube. Thus, compression, obstruction and wedging mechanisms of known systems can be eliminated.

There is a further continuing need for a magnetized acupuncture needle assembly which includes a magnetically charged acupuncture needle's handle and a magnetically charged needle guide tube to automatically attract the acupuncture needle's handle within the hollow center of the needle guide tube, as the needle is journalled or threaded in the tube. The magnetic charge of the handle and the tube are oppositely charged.

Thus, there is a continuing need for a magnetized acupuncture needle assembly which prevents or minimizes needle-to-tube contact in the hollow center of the needle guide tube so that the needle and its point remain sterile.

There is a still continuing need for a magnetized acupuncture needle assembly which is easy to thread.

### SUMMARY OF THE INVENTION

In view of the aforementioned problems, it is an object of the present invention to oppositely magnetically charge acupuncture needle assembly components so that the needle and the needle guide tube magnetically adhere together.

A further object of the present invention is to embed material, particles, powder or the like into the needle guide tube which is operable to magnetically attract or be magnetically attracted to the material of the needle handle.

A still further object of the present invention is to minimize or prevent needle-to-tube contact in the hollow center so that the needle and its needlepoint end remain sterile.

A still further object of the present invention is to automatically align the acupuncture needle essentially parallel to the longitudinal axis of the tube's hollow center to enhance the precision of penetration into the body tissue as the acupuncturist inserts the acupuncture needle.

A still further object of the present invention is to provide a magnetized acupuncture needle which is easily guided in and/or easily released from the hollow center of the needle guide tube.

A still further object of the present invention is to provide a magnetized acupuncture needle assembly which employs a combination of magnetic forces to attract portions (such as, the handle) of the acupuncture needle in the needle guide tube.

In view of the above objects, the present invention contemplates an acupuncture needle assembly comprising a needle guide tube having embedded therein material which is operable to magnetically attract or be magnetically attracted to material of an acupuncture needle to automatically hold the acupuncture needle in the needle guide tube.

In view of the above objects, the present invention contemplates a needle guide tube having embedded therein material which is operable to magnetically attract or be magnetically attracted to material of an acupuncture needle to automatically hold the acupuncture needle in the needle guide tube.

The present invention further contemplates a magnetized acupuncture needle comprising a magnetized needle handle; and an elongated and thin cylindrical member with a sharpened needlepoint end, the sharpened needlepoint end being constructed and arranged to penetrate body tissue wherein the magnetized handle emits a magnetic force operable to attract the needle handle within a needle guide tube.

The present invention further contemplates a magnetized acupuncture needle comprising a needle handle having embedded therein material which is operable to magnetically attract or be magnetically attracted to material of a needle guide tube; and an elongated and thin cylindrical member with a sharpened needlepoint end, the sharpened needlepoint end being constructed and arranged to penetrate body tissue.

The acupuncture needle's handle glows in the dark and includes phosphorescence material.

The present invention further contemplates a method of securing an acupuncture needle in a needle guide tube comprising the steps of: magnetically attracting, into a hollow center of the needle guide tube, the acupuncture needle; and, magnetically securing the acupuncture needle in the hollow center.

The magnetically attracting and holding steps include applying a combination of magnetic forces which attracts the needle handle to the needle guide tube.

The above and other objects and features of the present invention will become apparent from the drawings, the description given herein, and the appended claims.

### BRIEF DESCRIPTION OF THE DRAWING

For a further understanding of the nature and objects of the present invention, reference should be had to the following description taken in conjunction with the accompanying drawings in which like parts are given like reference numerals and, wherein:

FIG. 1A illustrates a cross sectional view of an acupuncture needle assembly of the prior art with a wedge member in a non-wedged state;

FIG. 1B illustrates a cross sectional view of an acupuncture needle assembly of the prior art with the wedge member in the wedged state;

FIG. 2A illustrates a cross sectional view of an alternate embodiment of an acupuncture needle assembly of the prior art with a wedge member in a non-wedged state;

FIG. 2B illustrates a cross sectional view of an alternate embodiment of an acupuncture needle assembly of the prior art with the wedge member in a wedged state;

FIG. 3A illustrates a cross sectional view of the acupuncture needle assembly of the present invention with the needle handle at a location which is out of the needle guide tube;

FIG. 3B illustrates a cross sectional view of the acupuncture needle assembly of the present invention with the needle handle at a location which is partially inside of the needle guide tube;

FIG. 4A illustrates a top view of an alternate embodiment of the needle guide tube of the present invention with the magnetic material at a top end of the needle guide tube;

FIG. 4B illustrates a perspective view of the embodiment of FIG. 4A;

FIG. 5 illustrates a perspective view of another alternate embodiment of the needle guide tube of the present invention with the magnetic material in helically wound strips around the needle guide tube;

FIG. 6A illustrates a top view of a still further alternate embodiment of the needle guide tube of the present invention with the magnetic material extending along the length for an arc section the needle guide tube;

FIG. 6B illustrates a perspective view of the embodiment of FIG. 6A;

FIG. 7 illustrates an alternate embodiment of the acupuncture needle of the present invention; and,

FIG. 8 illustrates a still further alternate embodiment of the acupuncture needle of the present invention.

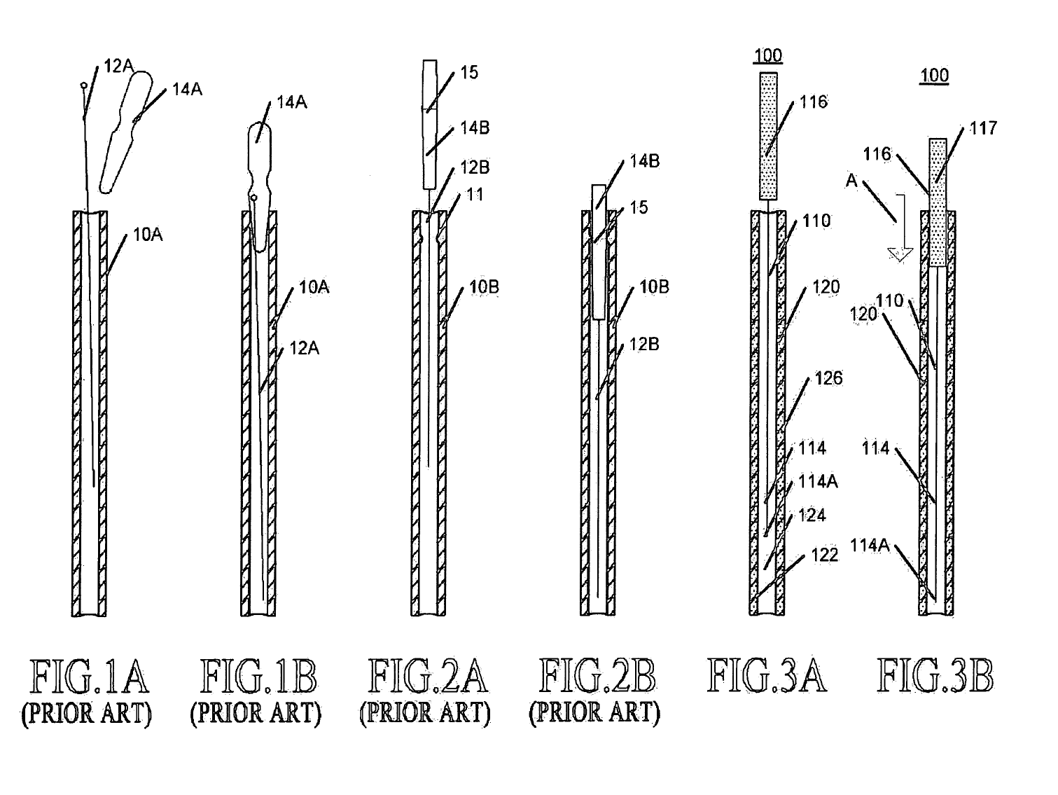

### DETAILED DESCRIPTION OF THE INVENTION

Referring now to the drawings, and particularly to FIGS. 3A and 3B, an acupuncture needle assembly according to the present invention is generally referenced by the numeral 100. The acupuncture needle assembly 100 comprises, in general, an acupuncture needle 110 having at least a needle handle 116. The assembly 100 also includes a needle guide tube 120. Generally, the needle guide tube 120 is made of a plastic material or non-metal material. However, in the present invention, the needle guide tube 120 has embedded in the plastic or non-metal body structure material, particles, powder or the like which is operable to magnetically attract or be magnetically attracted to the material of the needle handle 116.

Hence, magnetic attraction takes place between two oppositely charged magnetic particles or material. Thus, both the needle handle 116 and the needle guide tube 120 can have embedded or integrated into their respective body structures, magnetic particles so that the needle handle 116 is attracted to the needle guide tube 120.

Moreover, magnetic attraction take place between a magnetically charged material and a ferrous material. Thus, one of the needle handle 116 and the needle guide tube 120 would have embedded or integrated into their respective body structure, the magnetic material or particles while the other of the two includes ferrous material. Thus, in this arrangement, the magnetic attraction automatically takes place to attract the needle handle 116 and the needle guide tube 120 together.

In FIG. 3A, the acupuncture needle 110 is shown being journalled into the needle guide tube 120 with the handle 116 positioned outside of the tube 120. In the view of FIG. 3A, the handle 116 is being automatically attracted to the needle guide tube 120. FIG. 3B depicts the acupuncture needle 110 journalled into the needle guide tube 120 with at least a portion of the handle 116 also inside of the tube 120.

The needle guide tube 120 has an interior circumferential surface 122 which forms a hollow center 124, the hollow center 124 having a longitudinal axis. At least the interior circumferential surface 122 of the needle guide tube 120 includes material, particles, powder or the like which is operable to attract to or be attracted to material in the acupuncture needle 110. The acupuncture needle 110 is attracted, in the direction of ARROW A, into the hollow center 124. In the exemplary embodiment, a magnetic force is emitted approximately 360°. from the magnetized acupuncture needle 110. Nevertheless, the magnetic force may be emitted in the range of 0° to 360°.

As can be appreciated, the magnetic force automatically holds the acupuncture needle 110 within the hollow center 124 by virtue of its adherence to a ferrous or an oppositely charged magnetic material embedded in the structure of the needle guide tube 120. The magnetic force automatically attracts the acupuncture needle 110 to the hollow center 124 of the needle guide tube 120. The attraction of the magnetic force to ferrous material or an oppositely charged magnetic material allows the needle handle 116 to be automatically held in the hollow center 124 without any other means of securing the needle 110.

The acupuncture needle 110 comprises an elongated and thin cylindrical member 114 with a sharpened needlepoint end 114A, the sharpened needlepoint end 114A being constructed and arranged to penetrate body tissue. The magnetized acupuncture needle 110 further comprises a needle handle 116 at a distal end opposite the sharpened needlepoint end 114A.

At least the needle handle 116 is magnetized with the elongated and thin cylindrical member 114 and a sharpened needlepoint end 114A being non-magnetized. Nevertheless, the entire structure of the acupuncture needle 110 including the handle 116 and the elongated and thin cylindrical member 114 with a sharpened needlepoint end 114A may be magnetized.

In the exemplary embodiment, the needle handle 116 includes a plurality of magnetic or ferrous particles 117 (dots) embedded within a body or structure of the handle 116. The plurality of magnetic particles 117 (dots) are dispersed circumferentially around and along a length of the body of the handle 116. The dispersion may be evenly, may be in a collection of circumferential bands or vertical strips. Likewise, the needle guide tube includes a plurality of magnetic or ferrous particles 126 (dots) embedded within its body or structure.

Furthermore, the body or structure of the needle handle 116 includes phosphorescence material so that the handle 116 glows in the dark or illuminates in the dark. This feature helps the acupuncturist to see the glowing handle when performing services such as in a dim-lit room or in the dark. The glow in the dark feature also allows the acupuncturist to see the embedded handle within the tube 120.

The acupuncture needle assembly 100 takes advantage of an adherence force which automatically adheres the acupuncture needle 110 in the needle guide tube 120. This adherence is automatic and removable so that the holding force can be easily removed. The adherence force does not impede the ability of the acupuncturist to later guide the acupuncture needle 110 into the body tissue. The absence of wedging or other obstructions in the needle guide tube simplifies threading of the acupuncture needle 110. Moreover, guiding of the acupuncture needle 110 into the hollow center 124 is also easier.

The magnetic force is sufficient to automatically attract and hold the acupuncture needle 110 into the hollow center 124. However, when inserting the acupuncture needle 110 into the body tissue, the force or tapping by the acupuncturist as applied to handle 116 is sufficient to overcome the magnetic force. Thereby, the acupuncture needle 110 penetrates the body tissue. Hence, the insertion mode is not changed or made more difficult with the magnetic force. Instead, the insertion of the acupuncture needle 110 is made easier.

The sharpened needlepoint end 114A is generally sharpened to a radius in microns for adequate skin (body tissue) piercing or penetration as the acupuncture needle 110 is inserted. The needle handle 116 has a larger circumference then the elongated and thin cylindrical member 114.

In summary, the needle handle 116 may be magnetized to a first polarity and the needle guide tube 120 magnetized to a second polarity wherein the first and second polarities are attracted to each other. The elongated and thin cylindrical member 114 with the sharpened needlepoint end 114A may also be magnetized or magnetized in lieu of the handle 116.

While not wishing to be bound by theory, magnetic fields are used to treat joint and muscle aches. Thus, the magnetic field applied to the needle handle 116 may provided some added benefit after the needle is pierced in the body tissue.

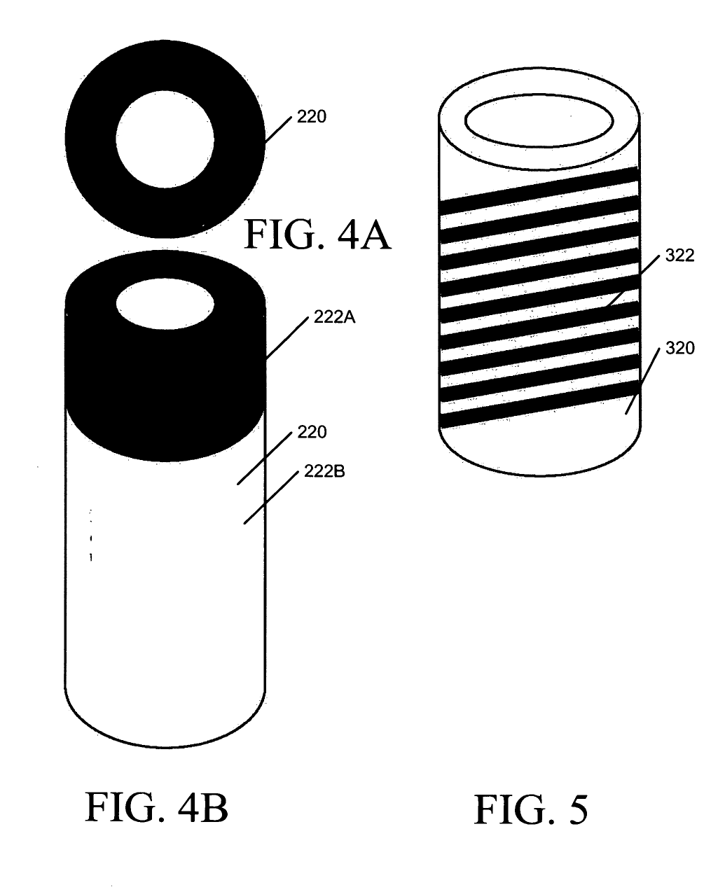

Referring now to FIGS. 4A and 4B an alternate embodiment of the needle guide tube 220 of the present invention is shown. The tube 220 has a top circumferential portion with the magnetic material 222A at a top end of the needle guide tube 220. The bottom remaining portion 222B of the needle guide tube 220 is made of plastic material. The top end of the needle guide 220 having the magnetic material 222A may be ⅓ of the length of the tube. Nevertheless, other lengths may be substituted.

With reference to FIG. 7, an alternate embodiment of the acupuncture needle 210 has a needle handle which includes a bottom end 217B comprised of magnetic or ferrous particles or material. The top portion 217A of the needle handle is non-magnetic and would glow in the dark. The elongated and thin cylindrical member of the needle 310 is made by metal denoted by the dotted line.

As can be readily seen, the acupuncture needle 210 would be easily released to the body tissue once the magnetic bottom end 217B of the needle handle clears the magnetic material 222A at a top end of the needle guide tube 220.

FIG. 5 illustrates a perspective view of another alternate embodiment of the needle guide tube 320 of the present invention with the magnetic material 322 arranged as a helically wound strip around the needle guide tube 320.

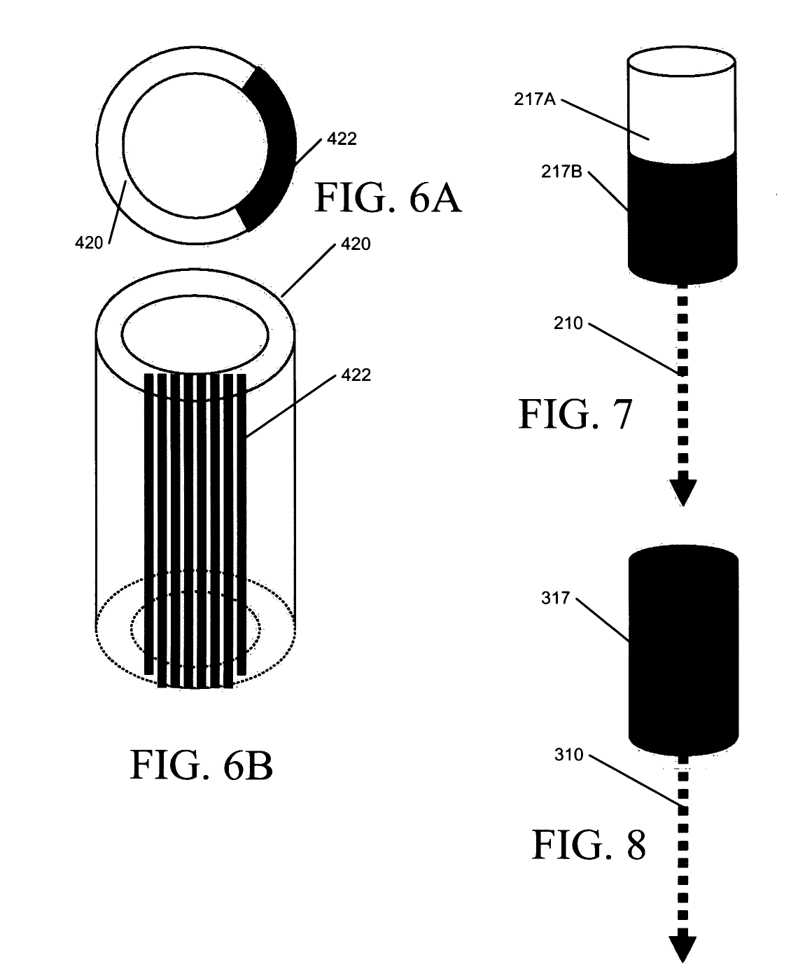

FIGS. 6A and 6B illustrates a top view of a still further alternate embodiment of the needle guide tube 420 of the present invention with the magnetic material 422 in the form of strips extending along the length for an arc section the needle guide tube 420. In this embodiment, the arc may be ⅓ of the circumference of the tube 420.

FIG. 8 illustrates a still further alternate embodiment of the acupuncture needle 310 of the present invention wherein the needle handle is made of magnetic material 317.

The foregoing description of the embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the, invention in various embodiments and with various modifications as are suited to the particular use contemplated. It is intended that the scope of the invention be defined by the claims appended hereto, and their equivalents.

What is claimed is:

1. A magnetized acupuncture needle assembly comprising:
an acupuncture needle having a portion which has a first material; and,
a needle guide tube having a body with an interior circumferential surface forming a hollow center and having embedded in the body a second material wherein the second material is operable to magnetically attract or be magnetically attracted to the first material to automatically attract the acupuncture needle in the needle guide tube.

2. The assembly according to claim 1, wherein the first material is magnetic with a first polarity and the second material is magnetic with a second polarity opposite the first polarity.

3. The assembly according to claim 1, wherein the first material and the second material automatically hold the acupuncture needle within the hollow center of the needle guide tube.

4. The assembly according to claim 1, wherein one of the first material and the second material is magnetic while the other of the first material and the second material is ferrous material.

5. The assembly according to claim 1, wherein the acupuncture needle comprises:
an elongated and thin cylindrical member with a sharpened needlepoint end, the sharpened needlepoint end being constructed and arranged to penetrate body tissue; and
a needle handle at a distal end opposite the sharpened needlepoint end.

6. The assembly according to claim 5, wherein the first material includes a plurality of magnetic particles embedded within a body of the needle handle.

7. The assembly according to claim 6, wherein the plurality of magnetic particles are dispersed circumferentially around and along a length of the body of the needle handle.

8. The assembly according to claim 7, wherein the body of the needle handle includes phosphorescence material.

9. The assembly according to claim 5, wherein the second material includes a plurality of magnetic particles embedded within the body of the needle guide tube.

10. The assembly according to claim 9, wherein the plurality of magnetic particles are dispersed circumferentially around and along a length of the body of the needle handle.

11. The assembly according to claim 9, wherein the plurality of magnetic particles are dispersed circumferentially around and along a bottom portion of the body of the needle handle.

12. The assembly according to claim 11, wherein the second material is a positioned at an upper portion of the needle guide tube.

13. The assembly according to claim 1, wherein the second material is a helically wound strip around the needle guide tube.

14. The assembly according to claim 1, wherein the second material is a longitudinal strip along a side of the needle guide tube.

15. A magnetized acupuncture needle comprising:
a needle handle having embedded therein material which is operable to magnetically attract or be magnetically attracted to material of a needle handle; and
an elongated and thin cylindrical member with a sharpened needlepoint end, the sharpened needlepoint end being constructed and arranged to penetrate body tissue.

16. The needle according to claim 15, wherein the needle handle includes phosphorescence material.

17. The needle according to claim 15, wherein the material emits a magnetic force within a range of 0°-360°.

18. The needle according to claim 15, wherein the material includes a plurality of magnetic particles dispersed circumferentially around and along a length of the handle.

19. The needle according to claim 18, wherein the plurality of magnetic particles are dispersed circumferentially around and along a bottom portion of the needle handle.

20. A method of securing an acupuncture needle in a needle guide tube comprising the steps of:
magnetically attracting, in a hollow center of the needle guide tube, the acupuncture needle; and,
magnetically securing the acupuncture needle in the hollow center.

21. The method according to claim 20, further comprising:
emitting a magnetic force from a needle handle of the acupuncture needle, during the magnetically attracting step.

22. The method according to claim 21, further comprising the step of:
emitting a magnetic force from the needle guide tube during the magnetically attracting step.

23. The method according to claim 20, further comprising the step of:
illuminating a needle handle of the acupuncture needle in a dark or a dim lit room.

24. A needle guide tube comprising non-metal material forming a tubular body with a hollow center, the tubular body having embedded therein particles which are operable to magnetically attract or be magnetically attracted to material of an acupuncture needle.

25. The tube according to claim 24, wherein the particles are positioned at an upper portion of the tubular body.

26. The tube according to claim 24, wherein the particles are arranged as a helically wound strip around the tubular body.

27. The tube according to claim 24, wherein the particles are arranged as longitudinal strips along a side of the tubular body.
